(19) United States
(12) Patent Application Publication
(10) Pub. No.: US 2004/0265617 A1
(43) Pub. Date: Dec. 30, 2004

(54) Disk substrate for a perpendicular magnetic recording medium and perpendicular magnetic recording disk

(75) Inventors: Kim Kong, Akishima-shi (JP); Junichi Horikawa, Kofu-shi (JP)

Correspondence Address: SUGHRUE MION, PLLC, 2100 PENNSYLVANIA AVENUE, N.W., SUITE 800, WASHINGTON, DC, 20037, US

(73) Assignee: HOYA CORPORATION

(21) Appl. No.: 10/795,383

(22) Filed: Mar. 9, 2004

(30) Foreign Application Priority Data
Mar. 10, 2003 (JP) 64002/2003

(51) Int. Cl. A61B 8/00; G11B 5/56; G11B 21/24; H01F 1/00

(52) U.S. Cl. 428/611; 360/294

(57) ABSTRACT

A disk substrate for a perpendicular magnetic recording medium is formed, on a substrate, with a soft magnetic underlayer and a nonmagnetic underlayer. The nonmagnetic underlayer is made of an amorphous material containing Cr as a main component. A perpendicular magnetic recording disk is constituted by forming a perpendicular magnetic recording layer on the magnetic disk substrate.

This application claims priority to Japanese Patent Application No. 2003-64002, the disclosure of which is incorporated herein by reference.

### BACKGROUND OF THE INVENTION

The present invention relates to a magnetic disk substrate suitable for a perpendicular magnetic recording disk or the like that is mounted in a HDD (hard disk drive) or the like of a perpendicular magnetic recording system and that enables an increase in recording density, and a perpendicular magnetic recording disk.

The information society in recent years has continued to make rapid progress, and an information recording capacity exceeding 60 Gbytes has come to be required per 2.5-inch magnetic disk in a magnetic recording device represented by a HDD (hard disk drive).

For complying with such a requirement, it is required to realize an information recording density exceeding 100 Gbits per square inch (100 Gbit/inch) in the magnetic disk. For carrying out stable recording/reproduction at such a high recording density, it is preferable to employ a perpendicular magnetic recording system as a magnetic recording/reproducing system. In particular, the perpendicular magnetic recording system is highly resistive to a thermal fluctuation error due to thermal magnetic aftereffect and therefore is especially preferable in a high recording density region.

For adapting a magnetic disk to the perpendicular magnetic recording system, there is required a design concept largely different from that of a magnetic disk for the in-plane magnetic recording system that is widely used at present.

A perpendicular magnetic recording disk is preferably a so-called double-layer perpendicular magnetic recording disk having, on a substrate, a soft magnetic underlayer made of a soft magnetic substance and a perpendicular magnetic recording layer made of a hard magnetic substance. In the double-layer perpendicular magnetic recording disk, a suitable magnetic circuit can be formed across a magnetic head, the perpendicular magnetic recording layer, and the soft magnetic layer upon magnetic recording so that the soft magnetic layer helps with the magnetic recording on the perpendicular magnetic recording layer.

On the other hand, it is also preferable to form a nonmagnetic underlayer between the soft magnetic underlayer and the perpendicular magnetic recording layer.

As such a double-layer perpendicular magnetic recording disk, there is known, for example, a perpendicular magnetic recording medium as described in Japanese Patent Application Publication (JP-A) No. 2002-74648, which will be hereinafter referred to as Reference 1.

In a magnetic recording medium, it is essential to reduce magnetization transition noise of a magnetic recording layer for improving a S/N ratio of a recorded/reproduced signal. It is known that the magnetization transition noise is generally affected by orientation, shapes, grain sizes, and dispersion thereof, of magnetic grains forming the magnetic recording layer.

Further, it is known that the S/N ratio is also affected by influence of magnetic interactions such as exchange interactions existing between the magnetic grains. When the influence of the magnetic interactions existing between the magnetic grains is large, it is possible that magnetization inversion between recording bits is impeded and, in particular, as the information recording density increases, suitable recording/reproduction is prevented.

In order to suppress such magnetic interactions, grain boundary portions are normally formed between the magnetic grains to thereby interrupt the magnetic interactions.

For example, when the magnetic recording layer is a hard magnetic layer of a Co-based alloy suitable for an increase in recording density, it is desirable to form grain boundaries in a hcp crystal structure of the Co-based alloy. However, there has been a problem that when the Co-based alloy is used as a perpendicular magnetic recording layer, it is difficult to form suitable grain boundaries.

According to the study by the present inventors, this is considered to be caused by the fact that, in the in-plane magnetic recording disk, since the hcp crystal structure of the Co-based alloy is oriented in the plane of the disk, a material to form grain boundary portions is prevented from flowing out to the exterior of the magnetic recording layer so that grain boundaries tend to be formed and grains also tend to be small, while, in the perpendicular magnetic recording medium, since the hcp crystal structure is oriented perpendicular to the plane of the disk, a material to form grain boundary portions tends to diffuse and flow out in a direction perpendicular to the magnetic recording layer, i.e. into a layer (e.g. a soft magnetic underlayer or a nonmagnetic underlayer) on the lower side of the magnetic recording layer, or into a layer (e.g. a protective layer) on the upper side thereof so that suitable grain boundaries are difficult to form.

Consequently, it is difficult to further increase the S/N ratio of the perpendicular magnetic recording medium, which is impeding measures to achieve an increase in recording density.

Further, a recording/reproducing error due to deterioration of the soft magnetic underlayer has become a large problem in the perpendicular magnetic recording disk. In order to improve the S/N ratio of the perpendicular magnetic recording layer, an attempt has been made to heat the substrate before forming the perpendicular magnetic recording layer. This aims to improve the S/N ratio by forming the perpendicular magnetic recording layer on the heated substrate to control the foregoing orientation, shapes, grain sizes, and dispersion thereof, of the magnetic grains.

However, a soft magnetic material forming the soft magnetic layer is often such a material that is small in corrosion resistance and therefore the property of the soft magnetic layer is often degraded upon heating. When the property of the soft magnetic layer is degraded, it is possible that formation of a suitable magnetic circuit across the magnetic head, the perpendicular magnetic recording layer, and the soft magnetic layer is prevented to lower the S/N ratio. Further, it is considered that when the perpendicular magnetic recording layer is formed on the heated substrate, the material forming the grain boundary portions is aided to diffuse and flow out, which, therefore, may lower the S/N ratio instead.

In consequence, conventionally, it has been difficult to stably mass-produce (produce in large quantity) perpendicular magnetic recording disks that can ensure high S/N ratios and therefore it has been prevented to give high-level quality assurance to shipping products.

### SUMMARY OF THE INVENTION

It is therefore an object of the present invention to provide a magnetic disk substrate suitable for a perpendicular magnetic recording medium, which can realize a perpendicular magnetic recording disk with a high S/N ratio to thereby obtain the perpendicular magnetic recording disk with a stable quality, which has a resistance against a thermal fluctuation error and is suitable for an increase in recording density.

Further, it is another object of the present invention to provide a perpendicular magnetic recording disk using the foregoing magnetic disk substrate.

In order to accomplish the foregoing objects, the present inventors have paid attention to a material of a nonmagnetic underlayer on the lower side of a perpendicular magnetic recording layer and, as a result of assiduous studies, they have found that a high S/N ratio can be realized by a perpendicular magnetic recording disk using the nonmagnetic underlayer made of an amorphous material containing Cr as a main component, and have reached the completion of the present invention based on the acquired knowledge.

According to one aspect of the present invention, there is provided a magnetic disk substrate having a soft magnetic underlayer and a nonmagnetic underlayer that are formed over a substrate. In the, magnetic disk substrate, the nonmagnetic underlayer is made of an amorphous material containing Cr as a main component.

Further, according to another aspect of the present invention, there is provided a magnetic disk substrate being used in a magnetic recording medium.

Further, according to still another aspect of the present invention, there is provided a magnetic disk substrate. In the magnetic disk substrate, the magnetic recording medium is a perpendicular magnetic recording medium.

Moreover, according to yet another aspect of the present invention, there is provided a perpendicular magnetic recording disk using said magnetic disk substrate. In the perpendicular magnetic recording disk, the perpendicular magnetic recording layer is formed on the magnetic disk substrate.

### BRIEF DESCRIPTION OF THE DRAWINGS

FIG. 1 is a sectional view showing a layer structure of one embodiment of a perpendicular magnetic recording disk according to the present invention; and

FIG. 2 is a diagram showing X-ray diffraction patterns of a CrTiTa film (Example) and a Ru film (Comparative Example).

### DESCRIPTION OF THE PREFERRED EMBODIMENT

The present invention will be further described in detail.

The present invention has the following structure.

First, in a structure 1, a disk substrate for a perpendicular magnetic recording medium is formed, on a substrate, with a soft magnetic underlayer and a nonmagnetic underlayer, and the foregoing nonmagnetic underlayer is made of an amorphous material containing Cr as a main component.

Then, in a structure 2, the foregoing nonmagnetic underlayer is made of an amorphous alloy material containing Cr and Ta in the disk substrate for the perpendicular magnetic recording medium of the foregoing structure 1.

Further, in a structure 3, the foregoing nonmagnetic underlayer is made of an amorphous alloy material containing Cr and Ta and further containing at least one of Ti and Ni in the disk substrate for the perpendicular magnetic recording medium of the foregoing structure 2.

Further, in a structure 4, the foregoing soft magnetic underlayer is made of a Co-based amorphous alloy material in the disk substrate for the perpendicular magnetic recording medium according to any of the structures 1 to 3.

Further, in a structure 5, an amorphous nonmetal intermediate layer is formed between the foregoing soft magnetic underlayer and nonmagnetic underlayer in the disk substrate for the perpendicular magnetic recording medium according to any of the structures 1 to 4.

Further, in a structure 6, a perpendicular magnetic recording disk is characterized in that a perpendicular magnetic recording layer is formed on the disk substrate for the perpendicular magnetic recording medium according to any of the structures 1 to 5.

As described in the structure 1, the disk substrate for the perpendicular magnetic recording medium of the present invention is formed, on the substrate, with the soft magnetic underlayer and the nonmagnetic underlayer, and the nonmagnetic underlayer is made of the amorphous material containing Cr as the main component. Further, the perpendicular magnetic recording disk can be obtained by forming the perpendicular magnetic recording layer on the disk substrate for the perpendicular magnetic recording medium.

The perpendicular magnetic recording disk having the perpendicular magnetic recording layer formed on the nonmagnetic underlayer made of such a Cr-based amorphous material can provide a high S/N ratio and contribute to an increase in recording density.

Amorphous referred to in the present invention represents a substance having no long-range order as defined in crystallography, for example, a complete amorphous substance or an amorphous substance containing nanocrystals. Inasmuch as there is no long-range order, the sharp peak resulted from a crystalline structure is not observed in an X-ray diffraction image.

The nonmagnetic underlayer exhibits an action of promoting crystal orientation of the perpendicular magnetic recording layer in a direction perpendicular to the plane of the substrate, and an action of promoting fineness of the perpendicular magnetic recording layer.

The material of the nonmagnetic underlayer in the present invention is the amorphous material containing Cr as the main component and, particularly the Cr-based amorphous alloy material is preferable. Such a Cr-based alloy is preferable because it vigorously exhibits an action of controlling a crystal axis (c-axis) of, for example, a CoPt-based perpendicular magnetic recording layer having a hcp crystal structure to be oriented in the perpendicular direction so that the hcp structure can grow finely and uniformly.

Specifically, by the use of the nonmagnetic underlayer of the Cr-based amorphous alloy, growing sites of nuclei for promoting the perpendicular orientation and fineness of the perpendicular magnetic recording layer can be formed so that the hcp crystal structure of the perpendicular magnetic recording layer can grow finely and uniformly, and suitable grain boundaries for suppressing magnetic interactions between magnetic grains can be formed by Cr diffusion from the nonmagnetic underlayer into the perpendicular magnetic recording layer. As a result thereof, the width of zigzag domain walls can be made small to reduce noise by nanocrystallization of the perpendicular magnetic recording layer and further the magnetic interactions between the magnetic grains can be suitably interrupted, and therefore, the high S/N ratio can be realized.

In the present invention, such a Cr-based amorphous alloy material is preferably the amorphous alloy material containing Cr and Ta. Cr alone usually forms a bcc crystal structure. On the other hand, by adding Ta to Cr to form an alloy, nanocrystals can be formed to achieve a suitable amorphous structure. The content of Ta preferably falls within a range of 5 at % to 50 at %. When the content of Ta is less than 5 at %, it is difficult to obtain an excellent amorphous structure, which is thus not preferable. On the other hand, when it exceeds 50 at %, it is possible that the action of promoting the perpendicular orientation and fineness of the perpendicular magnetic recording layer and the action of forming the grain boundaries for suppressing the magnetic interactions between the magnetic grains by Cr diffusion from the nonmagnetic underlayer into the perpendicular magnetic recording layer can not be sufficiently achieved, which is thus not preferable.

Moreover, it is preferably the amorphous alloy material containing Cr and Ta and further containing at least one of Ti and Ni. By the inclusion of Ti or Ni, the actions of forming the suitable amorphous structure and forming the growing sites of the perpendicular magnetic recording layer can be further promoted.

The film thickness of such a nonmagnetic underlayer is preferably 2 nm to 30 nm. When the film thickness of the nonmagnetic underlayer is less than 2 nm, the action of controlling the crystal axis of the perpendicular magnetic recording layer is insufficient, while, when it exceeds 30 nm, the size of the magnetic crystal grains forming the perpendicular magnetic recording layer is enlarged to increase noise, which is thus not preferable.

In the present invention, the soft magnetic underlayer for suitably adjusting a magnetic circuit of the perpendicular magnetic recording layer is provided on the substrate. This soft magnetic underlayer is an amorphous soft magnetic underlayer and is made of a soft magnetic substance having a soft magnetic property. As a material of the soft magnetic underlayer in the present invention, the Co-based amorphous alloy material is preferable and particularly a CoZr-based alloy material is preferable. Specifically, use can be made of a CoZrTa-based alloy, a CoNbZr-based alloy, a CoFeB-based alloy, or the like.

In the present invention, it is preferable to provide the amorphous nonmetal intermediate layer between the foregoing soft magnetic underlayer and nonmagnetic underlayer provided on the substrate. This nonmetal intermediate layer exhibits an action of protecting the soft magnetic underlayer located below and an action of promoting fineness of the nonmagnetic underlayer located above, and is an amorphous nonmetal nonmagnetic layer. As a material of the nonmetal intermediate layer in the present invention, amorphous carbon or the like, for example, is preferable.

In the present invention, although the substrate is not particularly limited, use can be made of a glass substrate or a metal substrate of an aluminum alloy or the like. When the glass substrate having a high smoothness is used, the flying height of a magnetic recording head can be reduced, which is thus particularly preferable.

In the present invention, it is preferable that the perpendicular magnetic recording layer be a CoPt-based perpendicular magnetic recording layer. Further, it is preferable that a crystal structure of the perpendicular magnetic recording layer be a hcp crystal structure. When the CoPt-based magnetic layer having the hcp crystal structure is used as the perpendicular magnetic recording layer, an easy magnetization axis of the perpendicular magnetic recording layer can be oriented perpendicular to the plane of the substrate by orienting a c-axis of the hcp crystal structure perpendicular thereto.

The foregoing CoPt-based perpendicular magnetic recording layer has a high coercive force Hc so that a magnetization reversal nucleation magnetic field Hn can have a small value of less than zero to thereby improve a resistance against thermal fluctuation, which is thus preferable.

In the perpendicular magnetic recording layer of the present invention, the content of Pt is preferably 10 at % to 25 at %, wherein 12 at % to 20 at % is particularly desirable. When the content of Pt is less than 10 at %, an anisotropic magnetic field Hk is reduced to lower the thermal fluctuation resistance, which is thus not preferable. On the other hand, when it exceeds 25 at %, it is possible that a stacking defect relative to a fcc crystal structure occurs, which is thus not preferable.

Particularly, in case of containing at least one kind of element from B, Nb, Zr, and Hf, since it exhibits an action of finely forming magnetic crystal grains constituting the perpendicular magnetic recording layer, it is suitable for an increase in recording density. Further, in the perpendicular magnetic recording layer of the present invention, the content of at least one kind of element selected from B, Nb, Zr, and Hf is preferably 1 at % to 20 at %, wherein 1 at % to 10 at % is particularly desirable. When the content of the element is less than 1 at %, the action of finely forming the magnetic crystal grains is lowered, which is thus not preferable, while, when it exceeds 20 at %, the perpendicular orientation of the perpendicular magnetic recording layer is degraded, which is thus not preferable.

Further, in the present invention, it is also preferable that the perpendicular magnetic recording layer contain Cr. By the inclusion of Cr in the perpendicular magnetic recording layer conjointly with Cr diffusion from the nonmagnetic underlayer, Cr can segregate to grain boundary portions of the magnetic crystal grains. Consequently, the suitable grain boundary portions by Cr can be formed between the magnetic grains so that it is possible to suppress the magnetic interactions between the magnetic crystal grains to thereby contribute to an increase in recording density.

When the perpendicular magnetic recording layer contains Cr, the content thereof is preferably 10 at % to 25 at % and more preferably 13 at % to 22 at %. When the content of Cr falls within the foregoing range, the formation of suitable boundaries between the magnetic crystal grains is facilitated. On the other hand, when the content of Cr exceeds 25 at %, it is possible that there is observed lowering in thermal fluctuation resistance due to reduction in anisotropic magnetic field Hk, which is thus not preferable.

In the present invention, it is preferable to provide a protective layer on the perpendicular magnetic recording layer. By providing the protective layer, it is possible to protect the surface of the magnetic disk from the magnetic recording head flying over the magnetic disk. As a material of the protective layer, a carbon-based protective layer, for example, is preferable. Further, the film thickness of the protective layer is preferably about 3 nm to 7 nm.

In the present invention, it is preferable to further provide a lubrication layer on the foregoing protective layer. By providing the lubrication layer, it is possible to prevent abrasion between the magnetic recording head and the magnetic disk to thereby improve durability of the magnetic disk. As a material of the lubrication layer, PFPE (perfluoropolyether), for example, is preferable. Further, the film thickness of the lubrication layer is preferably about 0.5 nm to 1.5 nm.

Now, an embodiment of the present invention will be described with reference to the drawings.

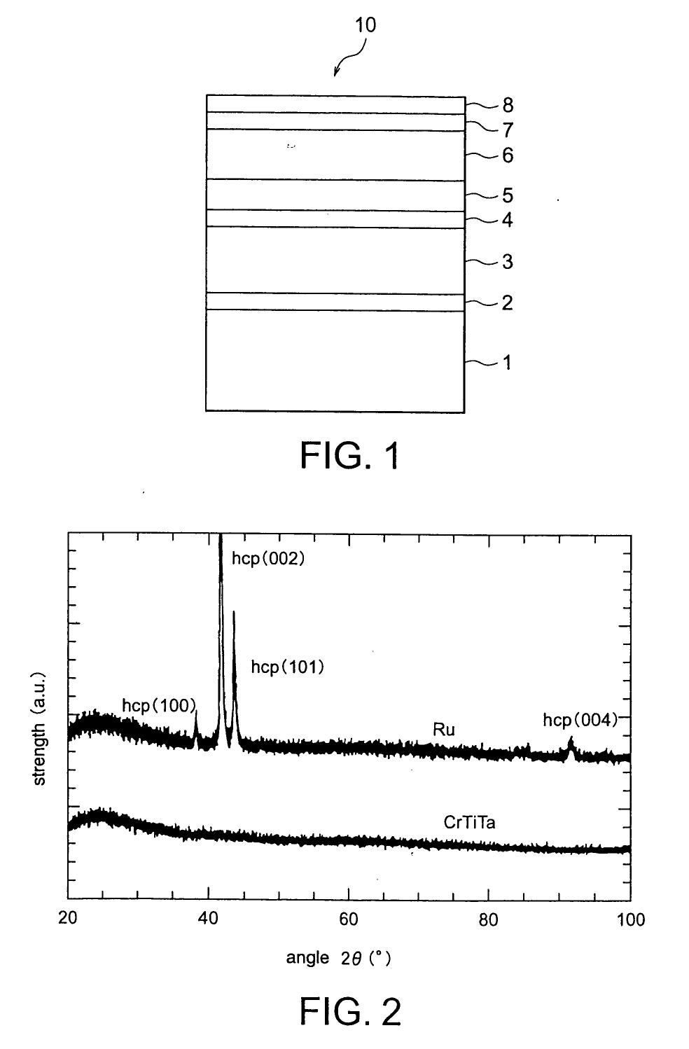

In FIG. 1, a perpendicular magnetic recording disk 10 according to this embodiment comprises an adhesion layer 2, a soft magnetic underlayer 3, a nonmetal intermediate layer 4, a nonmagnetic underlayer 5, a perpendicular magnetic recording layer 6, a protective layer 7, and a lubrication layer 8, which are stacked on a glass disk 1 in the order named.

The glass disk 1 is a glass disk made of chemically strengthened amorphous aluminosilicate glass.

The adhesion layer 2 exhibits an action of reinforcing an adhesion of the soft magnetic underlayer 3 relative to the glass disk 1.

The soft magnetic underlayer 3 is an amorphous alloy soft magnetic underlayer and is made of a soft magnetic substance having a soft magnetic property.

The nonmetal intermediate layer 4 is an amorphous nonmetal nonmagnetic layer and exhibits an action of protecting the soft magnetic underlayer 3 and an action of promoting fineness of the nonmagnetic underlayer 5.

The nonmagnetic underlayer 5 is a nonmagnetic underlayer made of an amorphous material containing Cr as a main component in the present invention and exhibits an action of promoting perpendicular orientation of the perpendicular magnetic recording layer 6 and an action of promoting fineness thereof.

The perpendicular magnetic recording layer 6 is a hcp crystal structure alloy hard magnetic recording layer. An easy magnetization axis is oriented perpendicular to the plane of the disk.

The protective layer 7 is a layer for protecting the perpendicular magnetic recording disk 10 from an impact of a magnetic head (not illustrated). Further, the lubrication layer 8 is a layer for relaxing the impact of the magnetic head (not illustrated).

In this embodiment, a magnetic disk substrate for perpendicular magnetic recording medium according to the present invention is constituted by forming, on the glass disk 1, the adhesion layer 2, the soft magnetic underlayer 3, the nonmetal intermediate layer 4, and the nonmagnetic underlayer 5.

Hereinbelow, specific examples of the present invention will be described.

### EXAMPLE 1

A glass disk base member was prepared by forming amorphous aluminosilicate glass into a disc shape by direct pressing. This glass disk base member was successively subjected to grinding, polishing, and chemical strengthening to thereby obtain a smooth nonmagnetic glass disk 1 in the form of a chemically strengthened glass disk.

The surface roughness of the principal surface of the glass disk 1 was measured by an AFM (atomic force microscope), resulting in a smooth surface profile having Rmax of 4.8 nm and Ra of 0.42 nm. Hereupon, Rmax and Ra follow the Japanese Industry Standard (JIS B 06 01).

By the use of a single-wafer stationary opposed-type film forming apparatus having been evacuated, an adhesion layer 2, a soft magnetic underlayer 3, and a nonmetal intermediate layer 4 were successively formed as films on the obtained glass disk 1 in an Ar atmosphere by the DC magnetron sputtering method.

UP The adhesion layer 2 was formed as a film by the use of a Ti target so as to be a Ti layer of 20 nm. The soft magnetic underlayer 3 was formed as a film by the use of a CoZrTa target so as to be a CoZrTa (Co: 88 at %, Zr: 5 at %, Ta: 7 at %) amorphous alloy layer of 300 nm. This CoZrTa alloy is a soft magnetic substance exhibiting a soft magnetic property. Further, the nonmetal intermediate layer 4 was formed as a film by the use of a graphite target so as to be a nonmagnetic nonmetal amorphous carbon layer (film thickness 1 nm).

The surface roughness of the disk obtained by thus forming the films up to the nonmetal intermediate layer 4 on the glass disk 1 was similarly measured by the AFM, resulting in a smooth surface profile having Rmax of 5.1 nm and Ra of 0.48 nm.

Further, the magnetic properties of the obtained disk were measured by a VSM (vibrating sample magnetometer), resulting in a coercive force (Hc) of 2 oersteds (Oe) and a saturation magnetic flux density of 810 emu/cc, thus exhibiting the suitable soft magnetic property.

The disk obtained above was heated in the film forming apparatus having been successively evacuated, and a nonmagnetic underlayer 5, a perpendicular magnetic recording layer 6, and a protective layer 7 were successively formed as films in the Ar atmosphere by the DC magnetron sputtering method.

The foregoing heating was carried in a vacuum at 275° C. for 9 seconds while the disk is retained.

The nonmagnetic underlayer 5 was formed as a film by the use of a CrTiTa target so that there was formed a CrTiTa (Cr: 45 at %, Ti: 45 at %, Ta: 10 at %) amorphous alloy layer having a thickness of 15 nm.

A magnetic disk substrate according to the example of the present invention was obtained by thus forming the films up to the nonmagnetic underlayer 5 on the glass disk 1.

In order to examine a fine structure of the nonmagnetic underlayer 5, an analysis was made by the XRD (X-ray diffraction measurement) method. The X-ray diffraction measurement was carried out by the goniometer method using Cu Kα radiation. A result of the analysis is shown in FIG. 2. As shown in FIG. 2, it was confirmed that the sharp peak resulted from a crystalline structure was not observed and therefore the CrTiTa film of the nonmagnetic underlayer was amorphous.

Then, by using a target made of a hard magnetic substance being a CoCrPt alloy, the perpendicular magnetic recording layer 6 of 20 nm having a hcp crystal structure was formed as a film on the disk substrate. The perpendicular magnetic recording layer 6 was made of an alloy material of Co: 62.5 at %, Cr: 20 at %, and Pt: 17.5 at %.

Then, by the use of a mixed gas containing Ar and 30% hydrogen added thereto, a carbon target was subjected to sputtering to thereby form the protective layer 7 (film thickness 5 nm) made of hydrogenated carbon. By using hydrogenated carbon, the film hardness is improved so that the perpendicular magnetic recording layer 6 can be protected against an impact from the magnetic head.

Thereafter, a lubrication layer 8 made of PFPE (perfluoropolyether) was formed by the dip coating method. The film thickness of the lubrication layer 8 was 1 nm.

Through the foregoing production processes, the perpendicular magnetic recording disk 10 of this embodiment was obtained.

The orientation of the perpendicular magnetic recording layer 6 of the obtained perpendicular magnetic recording disk 10 was analyzed by the X-ray diffraction method, resulting in that it was oriented in a direction perpendicular to the plane of the magnetic disk.

Further, the obtained perpendicular magnetic recording disk 10 was analyzed by a transmission electron microscope (TEM), and it was confirmed that the soft magnetic underlayer 3 was amorphous wherein no long-range order was observed. Deterioration in quality due to heating was not observed in the soft magnetic underlayer 3.

Further, the magnetic properties of the obtained perpendicular magnetic recording disk 10 was evaluated by the VSM, resulting in a coercive force (Hc) of 4200 oersteds, a squareness ratio (residual magnetization (Mr)/saturation magnetization (Ms)) of 0.94, and a magnetization reversal nucleation magnetic field (Hn) of −1000 oersteds, thus exhibiting suitable magnetic properties. Further, the slope of a MH curve exhibited a suitable magnetic property of 1.2/4π.

As the magnetic properties, the coercive force and the squareness ratio are preferably as high in numerical value as possible, while the magnetization reversal nucleation magnetic field is preferably as small a value less than zero as possible. Further, the slope of the MH curve is preferably as close to 1.0/4π as possible. This is because, in theory, if it is 1.0/4π, the magnetic interactions are considered to be suppressed and not substantially effected.

Further, electromagnetic conversion properties of the obtained perpendicular magnetic recording disk 10 were measured, and a result suitable for the magnetic disk was obtained that the S/N ratio was 25.8 dB and the recording density was 100 Gbits/inch² or more.

The electromagnetic properties were measured in the following manner.

The measurement was carried out at a recording density of 780 kfci by the use of a R/W analyzer (GUZIK) and a magnetic head for the perpendicular magnetic recording system having a SPT element on the recording side and a GMR element on the reproducing side. In this event, the flying height of the magnetic head was 12 nm.

Further, the thermal fluctuation was also measured and no error was confirmed.

The results of the foregoing magnetic properties and the like are also shown collectively in Table 1 below.

### EXAMPLES 2 AND 3

In Example 1 as described above, the nonmagnetic underlayer 5 was formed as an amorphous alloy layer made of CrNiTa (Cr: 50 at %, Ni: 40 at %, Ta: 10 at %) (Example 2). By the use of a CrNiTa target, the CrNiTa alloy layer was formed to a thickness of 15 nm. An analysis was made by the X-ray diffraction measurement like in Example 1, and it was confirmed that the foregoing CrNiTa film was amorphous.

Further, in Example 1, the nonmagnetic underlayer 5 was formed as an amorphous alloy layer made of CrTa (Cr: 60 at %, Ta: 40 at %) (Example 3). By the use of a CrTa target, the CrTa alloy layer was formed to a thickness of 15 nm. An analysis was made by the X-ray diffraction measurement like in Example 1, and it was confirmed that the foregoing CrTa film was amorphous.

Perpendicular magnetic recording disks were obtained by the same production method as in Example 1 except that the materials of the nonmagnetic underlayers 5 were different as described above.

Results of analyzing and evaluating the obtained perpendicular magnetic recording disks like in Example 1 are shown in Table 1 below.

### COMPARATIVE EXAMPLES 1 AND 2

In Example 1, the nonmagnetic underlayer 5 was formed as an amorphous alloy layer made of NiZrTa (Ni: 40 at %, Zr: 50 at %, Ta: 10 at %) (Comparative Example 1). By the use of a NiZrTa target, the NiZrTa alloy layer was formed to a thickness of 15 nm.

Further, in Example 1, the nonmagnetic underlayer 5 was formed as a Ru layer (Comparative Example 2). By the use of a Ru target, the Ru layer was formed to a thickness of 15 nm. An analysis was made by the X-ray diffraction measurement like in Example 1, and it was confirmed that, as shown in FIG. 2, the sharp diffraction peaks were observed in an X-ray diffraction pattern and therefore the foregoing Ru film was crystalline (hcp crystal structure).

Perpendicular magnetic recording disks were obtained by the same production method as in Example 1 except that the materials of the nonmagnetic underlayers 5 were different as described above.

Results of analyzing and evaluating the obtained perpendicular magnetic recording disks like in Example 1 are shown in Table 1 below.

From the results of Table 1 below, it is understood that, in each perpendicular magnetic recording disk according to the embodiment of the present invention, the nonmagnetic underlayer provided under the perpendicular magnetic recording layer is made of the amorphous alloy material containing Cr as the main component to thereby realize the high S/N ratio. Further, each perpendicular magnetic recording disk of the embodiment exhibits the excellent result also in terms of the magnetic properties.

This is because, by the use of the Cr-based nonmagnetic underlayer, growing sites of nuclei for promoting the perpendicular orientation and fineness of the perpendicular magnetic recording layer are provided so that the hcp crystal structure of the CoCr-based alloy can grow finely and uniformly, and suitable grain boundaries for suppressing magnetic interactions between CoCr-based magnetic grains are formed by Cr diffusion from the nonmagnetic underlayer into the perpendicular magnetic recording layer. As a result thereof, it is considered that the width of zigzag domain walls can be made small to reduce noise by nanocrystallization of the perpendicular magnetic recording layer and further the magnetic interactions between the magnetic grains can be suitably interrupted, and therefore, the high S/N ratio is realized.

TABLE 1

| | | | magnetization | slope | | square- |
|---|---|---|---|---|---|---|
| | material of | coercive | reversal nuclea- | of MH | SIN | ness |
| | nonmagnetic | force | tion magnetic | curve | ratio | ratio |
| Table 1 | underlayer | Hc (Oe) | field Hn (Oe) | (× ¼π) | (dB) | Mr/Ms |
| Example 1 | CrTiTa | 4200 | −1000 | 1.2 | 25.8 | 0.94 |
| Example 2 | CrNiTa | 3900 | −300 | 1.2 | 25.8 | 0.93 |
| Example 3 | CrTa | 4000 | −500 | 1.3 | 26.0 | 0.92 |
| Comparative | NiZrTa | 3500 | −200 | 1.7 | 22.8 | 0.93 |
| Example 1 | | | | | | |
| Comparative | Ru | 4000 | 0 | 1.3 | 20.5 | 0.87 |
| Example 2 | | | | | | |

In contrast, the perpendicular magnetic recording disk of Comparative Example 1 using the nonmagnetic underlayer containing Ni as a main component has a very low S/N ratio and therefore can not comply with the demand for an increase-in recording density. Further, in the perpendicular magnetic recording disk of Comparative Example 2 using the crystalline nonmagnetic underlayer made of Ru, the S/N ratio is further lower as compared with even Comparative Example 1 and therefore it is totally impossible to realize an increase in recording density.

As described above in detail, according to the present invention, it is possible to realize a perpendicular magnetic recording disk with a high S/N ratio. And, it is possible to provide a magnetic disk substrate suitable for a perpendicular magnetic recording medium, which can realize with a stable quality such a perpendicular magnetic recording disk having the high S/N ratio, suitable for an increase in recording density, and having a resistance against a thermal fluctuation error.

What is claimed is:

1. A magnetic disk substrate having a soft magnetic underlayer and a nonmagnetic underlayer that are formed over a substrate, wherein said nonmagnetic underlayer is made of an amorphous material containing Cr as a main component.

2. A magnetic disk substrate according to claim 1, wherein said nonmagnetic underlayer is made of an amorphous alloy material containing Cr and Ta.

3. A magnetic disk substrate according to claim 2, wherein the nonmagnetic underlayer is made of an amorphous alloy material containing Cr and Ta and further containing at least one of Ti and Ni.

4. A magnetic disk substrate according to any one of claims 1 to 3, wherein said soft magnetic underlayer is made of a Co-based amorphous alloy material.

5. A magnetic disk substrate according to any one of claims 1 to 4, wherein an amorphous nonmetal intermediate layer is formed between said soft magnetic underlayer and said nonmagnetic underlayer.

6. A perpendicular magnetic recording disk comprising the magnetic disk substrate according to claim 1, wherein a perpendicular magnetic recording layer is formed on said nonmagnetic underlayer.

7. A magnetic disk substrate according to claim 1, wherein said substrate is formed by a glass disk, and an adhesion layer for reinforcing an adhesion of said soft magnetic underlayer is provided between said substrate and said soft magnetic underlayer.

8. A magnetic disk substrate according to claim 1, wherein at least one of a protective layer and a lubrication layer is provided at an uppermost layer.

9. A magnetic disk substrate according to claim 1, said magnetic disk substrate being used in a magnetic recording medium.

10. A magnetic disk substrate according to claim 9, wherein said magnetic recording medium is a perpendicular magnetic recording medium.

11. A perpendicular magnetic recording disk using the magnetic disk substrate according to claim 10, wherein said perpendicular magnetic recording layer is formed on said magnetic disk substrate.
